(19) United States
(12) Patent Application Publication
(10) Pub. No.: US 2005/0234564 A1
(43) Pub. Date: Oct. 20, 2005

(54) Enhanced-functionality prosthetic limb

(76) Inventors: Rainer Fink, College Station, TX (US); Sankar Pemmaraju, Keller, TX (US); Dennis Robbins, Richardson, TX (US); Robert W. Schwartz, Richardson, TX (US); Pradeep Shah, Dallas, TX (US); Tachung C. Yih, San Antonio, TX (US)

Correspondence Address: Michael G. Cameron, Suite 600, 2435 N. Central Expressway, Richardson, TX, 75080, US

(21) Appl. No.: 11/093,681

(22) Filed: Mar. 30, 2005

Related U.S. Application Data
(60) Provisional application No. 60/557,509, filed on Mar. 30, 2004.

(51) Int. Cl. A61F 2/58; A61F 2/56

(52) U.S. Cl. 623/62; 623/901; 623/64

(57) ABSTRACT

An enhanced-functionality prosthetic limb is disclosed, comprising a prosthetic hand or gripping device, and forearm, which may be body-powered or motor-powered, targeted primarily for pediatric use, attachable to the arm stump of a below-elbow, or above-elbow, with the inclusion of a prosthetic elbow, amputee individual, with incorporation of one or more of the following features: expandability in length or size to accommodate child growth or other expandability needs, grasp locking capability, individual finger locking capability, three degree of freedom wrist joint, dynamic tensional rotation control of wrist, dynamic grasp control to allow grasping of irregular objects, extended grasp for gripping larger objects, excessive force breakaway, and algorithms to support by user of care-provider adjustment of the prosthesis.

### CLAIM OF PRIORITY

This application claims priority to U.S. Provisional Patent Application Ser. No. 60/557,509, filed Mar. 30, 2004, entitled “Enhanced Functionality Prosthetic Arm,” the entire contents of which are incorporated herein by this reference.

### STATEMENT REGARDING FEDERALLY SPONSORED RESEARCH OR DEVELOPMENT

No federal grants or funds were used in the development of the present invention.

### FIELD OF THE INVENTION

This present invention relates to prosthetic devices, particularly prosthetic hands and forearms, targeted primarily for pediatric use.

### BACKGROUND OF THE INVENTION

Children often reject the use of prosthetic upper limbs due to a combination of factors, including limited functionality in everyday use, unsatisfactory cosmetic appearance of devices, such as size mismatch when the child grows, weight of the devices, and the tendency of devices to block sensory feedback from the end of the extremity.

There are two conventional categories of prosthetic technologies: body powered and myoelectric. Both technologies have significant disadvantages. Body powered limbs were developed well before WWII and have not improved significantly since. Two versions exist in the body-powered technology: normally open and normally closed. The normally open version has not been extensively used due primarily to the frustration caused by lack of grip maintenance when the muscle control is released, causing a child to drop whatever is being held. Therefore, most children are fitted with normally closed, body-powered prostheses. For children, the weight of the myoelectric limb is often too great, and as such is not utilized in the targeted population. In either case, due to the rapid growth of children, new limbs are required every one to two years. At a cost of $2,000 to $15,000, the cost burden is significant.

Most prosthetic limbs fitted for children are simply scaled down versions of adult limbs. However, children often have difficulty adapting to a prosthesis due to cosmetic issues and difficulty in using them in everyday activities. It would be desirable to improve the dexterity in a child with a limb loss. If a child is able to participate in athletic events, play with toys or participate in the performing arts, it will greatly improve the acceptance and cosmetic appeal of the prosthetic limb and benefit the child's overall self-esteem.

Three types of prostheses are generally prescribed for the population of single-upper-limb amputee individuals: passive, active body-powered, and active myoelectric. Each are priced between $2,000 and $15,000. There is little research on prosthesis use in these children, and there have been no major new developments in upper extremity prosthesis technology for twenty (20) years. The limited information available indicates that the best prosthesis for younger children may be the “low-tech” body-powered prosthesis.

Data indicates that congenital limb deficiencies occur in 1 in 3,864 live births in the United States with upper limb deficiencies occurring 1.6 times more often (1 in 6,379) than lower limb deficiencies. An infant born with an upper-limb deficiency should be fitted with a prosthesis at about the time the child begins sitting. When selecting a prosthesis for initial fitting, three problems are considered: the weight of the appliance, the rapid growth of the infant and the choice of an appropriate terminal device. Fabricating the prosthesis from polypropylene solves the problem of weight. It is also possible to heat polypropylene to alter its shape, thereby readily accommodating minor changes in prosthesis size necessitated by growth. Fabricating the prosthetic shell from polypropylene is relatively inexpensive.

Although the deficiency demographics span a wide range of ages, the age-related acceptance of prostheses in children is primarily due to functional capabilities in day-to-day activities. In addition, the acceptability of the prosthesis is related to the mode of amputation: whether the deficiency was caused by a traumatic event early in life, or was a congenital defect. With respect to acceptance of prostheses, non-traumatic deficiencies result in children that have well developed substitute sensor systems as well as methods for accomplishing most desired tasks. These children are often perfectly capable of functioning without the prosthetic device. For the prosthesis to be accepted, it must demonstrate a significant enhancement in performance over the child's innate capability. With traumatic deficiencies, the loss of a limb results in a strong desire to replace the existing capability and as such results in a more readily trainable patient. Understanding these constraints as well as the changes in requirements as the patient ages leads to the desire to develop new technology, which is age and task appropriate for the juvenile market. The present invention benefits both congenital and traumatic deficiencies.

Conventional prosthetic hands are either body-powered (actuated with shoulder motion against a harness and cable system), or electric-powered (motor driven and either controlled by surface electrodes that detect underlying muscle activity, or through various switches attached to a harness). These devices provide only two degrees of freedom, open and closed, and generally, grasp is achieved by the thumb coming together with the first two finger tips in what is known as a “three-jaw chuck” grasp.

Recent work by various companies has been focused primarily on single performance features such as wrist flexion/extension, versus an integrated approach to providing a wide range of functionality features in a single prosthesis.

Hooks are also utilized, and in fact are still the most commonly fitted. They come in many shapes and sizes, but in two basic open or close strategies. The first is voluntary opening, in which rubber bands or springs keep the grip surfaces closed and provide constant pressure against which the amputee pulls, through the harness and cable system, to open the device. By relaxing his arm, the amputee enables the hook to close on an object or item. Pressure at the “finger tips” is constant, and is determined by spring tension or the number of rubber bands used. An advantage of voluntary closing devices is that once the amputee has grasped an object, pressure is maintained and it is no longer necessary to exert force through the harness system. The other option is the opposite, voluntary closing, in which the hook generally remains open and the amputee's force generated through the harness acts to close the device. Voluntary closing devices have the added advantage of providing some sensory feedback through the harness and the ability to vary grip force. But in order to maintain grasp of an object the amputee must exert constant pressure through the harness. Some systems allow the amputee to keep the device closed at pre-set positions by pressing a ball bearing on the cable into a “cleat” mounted on the forearm. One or two voluntary hook-type prostheses that are commercially available allow the user to lock or unlock the hook tips either manually or by control through the harness.

Hooks are still considered among the most functional of terminal devices for prostheses. Traditionally, hooks have been valued for their ability to grasp small and large objects with precision. Also, hooks do not block the user's view of objects as prosthetic hands may do. Hooks have low cost, high durability, and appropriateness for labor and leisure activities. However, a frequently cited complaint by users of hooks is that they would prefer the devices to appear more hand-like, or that they would prefer to have better prosthetic hands in general. Recently, devices that are more “hook-like” in function have been designed to include cosmetic features that help them appear more like a hand. In effect, the thumb and first two fingers grasp via a harness and cable system, and the fourth and fifth “fingers,” are sculpted into the rubber or urethane to make them appear to have been flexed into the “palm” of the device.

The two body-powered strategies previously mentioned, voluntary opening and voluntary closing, are available in both hook and hand designs. Myoelectric hands that are controlled by two electrodes could be considered to be both voluntary opening and closing, in that the amputee can volitionally open and close the device as well as stop the system in any position. Recently, a prototype body-powered hook design was introduced that will purportedly allow both voluntary opening and closing options in one device by virtue of a lever the amputee can flip to convert from one option to the other.

There are many options and much ongoing research, but essentially all these devices and strategies either utilize the “three-jaw chuck” form of grasp, or are versions of hooks. The present invention will enable a prosthesis with the cosmetic appearance of a hand that would combine many of the desirable features of both hands and hooks. The body-powered option is most appropriate since its lower cost should make it accessible to far broader range of users, including children, and potential users in many non-industrialized countries where cultural influences create a strong preference for hands over hooks.

In determining which design features were preferable and absent in current body-powered hands on the market, several studies are available. First of all, body-powered devices still seem to be prescribed and used more often. Particularly for adults, function seems to take precedence over cosmetic appearance, even though there may be dissatisfaction with the appearance of many body-powered devices. Among children and their parents, devices that are both functional and hand-like in appearance are overwhelmingly preferred. Why this changes by adulthood is unclear, but is most likely related to the pragmatic acceptance by maturity of function over appearance. In 1988, LeBlanc surveyed manufacturers of terminal devices. They indicated a 72% use of hooks and a 28% use of hands. A study of 314 adult amputees in Ontario by Millstein et al (1986) indicated that a body-powered device, primarily a hook, was more often selected by adult laborers who needed devices that were functional, durable and rugged.

An epidemiologic overview of upper limb amputees by Atkins et. al. (1996) is probably the largest and most comprehensive survey ever done in the United States on this subject. The authors reviewed responses from close to 2,500 amputees of all ages. LeBlanc compared results of the Atkins study to his earlier one and concluded that the use of myoelectric hands had increased by 9%, which may indicate that advances in myoelectric hands have encouraged more amputees to select them. In the Atkins study, adult amputees were the most common users of body-powered prostheses (65%). The authors conclude that this seems to be related to the functional advantages of body-powered hooks for manual labor, construction and farming. Children, however, were the most frequent users of electric prostheses, presumably because of a preference by parents for devices that were more hand-like in appearance. A review of over 300 clinical cases (adults and children) by Billock (1986) indicated a similar trend. From his perspective, though features of body-powered hooks are often more functional, there is a strong social-psychological preference for devices that are hand-like in appearance and still offer function.

In the Atkins study, research priorities identified by patients who wore either myoelectric hands, body-powered hooks, or both, indicate some significant needs, both functional and cosmetic that are not being met by any single system. Users of body-powered systems indicated the following user-based research priorities: improved cable systems; more comfortable harnesses; gloves that are more durable and aesthetic; wrist movement, and control mechanisms that would require less visual attention, and would enable coordinated motions of two joints. Users of electric prostheses prioritized: better gloving material; more reliable and durable hands; batteries and electrodes; greater finger movement; less visual attention required; and improved wrist movement.

A recent survey by Shaperman et al (2003), of children with upper limb absences also asked clinics to identify priorities for future research and improvements to upper limb prostheses. In order of priority ranked by the clinics, these included: increased grip strength; improved appearance; easier operation and simpler to operate mechanisms; better ability to hold objects through wider opening and a better hold on cylindrical objects; lighter weight; improved wrist motion in both rotation and flexion; either less harnessing or more comfortable harnessing systems; better visibility of objects held in the hand; more durable gloves: passive hands with positional fingers; other “wish-list” items such as compliant gripping surfaces, better thumb positioning, 3-point grasp and palm flexibility.

### SUMMARY OF INVENTION

The present invention addresses the issue of functionality, size adjustment, and weight, as well as providing affordability, when compared to myoelectric devices. The design of the present invention allows children significantly improved utilization of their prostheses in many day-to-day activities that are at best very difficult using conventional prosthetic hands/arms. Activities of daily living, including participating in sports that involve hand usage such as fishing, golf, baseball, etc., pushing buttons, utilizing different modes of grasp, holding objects that are not symmetrical, and grasping over-sized objects are often not easily performed using current state-of-the-art body powered devices.

The present invention is intended to address such needs using state-of-the-art technology and design methods, resulting in a more functional, longer lasting and more affordable, prosthetic limb.

The present invention comprises a novel prosthetic limb, which includes many of the functional capabilities of the myoelectric prosthesis, with the advantages of being highly reliable as well as low-priced.

### BRIEF DESCRIPTION OF THE DRAWINGS

FIG. 1 is an illustration of the EPC limb of the present invention;

FIG. 2 is an illustration of an extension tube;

FIG. 3 is an illustration of an extended grasp;

FIG. 4 is an illustration of the dynamic balanced group; and

FIG. 5 is an illustration of a ball and socket joint.

### DETAILED DESCRIPTION OF THE PRESENT INVENTION

Following is a chart depicting design features of the present invention. The present invention achieves the research priorities and “wish list” items identified by both patients and clinicians in the studies cited herein.

| Features of Present Invention: | | Conventional Devices |
|---|---|---|
| 1. | Esthetically-pleasing, multi-function hand | Myoelectric hands, but all offer the |
| | (does not require interchange of multiple terminal | same basic 3-jaw chuck grasp. |
| | devices.) | |
| 2. | Individual finger control - fingers could be | None |
| | activated, de-activated, or locked. | |
| 3. | Compliant grasp action so as to accommodate | None. (Only accomplished by |
| | to various sizes of spherical, cylindrical, or | interchange of multiple terminal |
| | irregular shaped objects | devices. Some hooks include |
| | | contours and openings that fit certain |
| | | sized objects such as a rake or broom |
| | | handle.) |
| 4. | “Lockdown” feature - ability to grasp an | Myoelectric hands, voluntary opening |
| | object and forget about it. | hooks or hands, or VC hooks with |
| | | mechanical “lock-down features. |
| 5. | Safety “breakaway” feature so that grasp will | Most devices allow either volitional |
| | release in an emergency situation | release or provide that the springs, |
| | | rubber bands or motor will “give” at a |
| | | certain point. |
| 6. | Three degrees of wrist motion (rotation, | Multi-position wrists (in flexion and |
| | flexion/extension) | extension) are available, primarily for |
| | | hooks, but some exist for specific |
| | | hands. Most involve a button that has |
| | | to be switched, or a ratchet device of |
| | | some sort. A “flexi-wrist” for young |
| | | children wearing myoelectric hands |
| | | will bend under loading, but returns to |
| | | its original position. |
| 7. | Option to engage larger opening function in | None, particularly in children's sizes. |
| | order to grasp oversized objects. | Widest opening is afforded by larger |
| | | adult sized myoelectric hands, some |
| | | larger hooks, and the Otto Bock |
| | | “Greifer”. |
| 8. | Lighter weight versus myoelectric arms. | This has to take into account the |
| | | weight of both the hand and the |
| | | battery and control system. |
| | | Prostheses with body-powered hands |
| | | are generally lighter overall than |
| | | myoelectric prostheses. |
| 9. | Easily expandable up to 30% (arm and finger | No hand on the market is expandable. |
| | length and hand width) as the child grows | Growth accommodation is generally |
| | | accomplished by interchanging |
| | | components, or by lengthening and re- |
| | | covering the forearm. Cables and |
| | | harnesses generally include enough |
| | | slack to be adjusted for 18 months or |
| | | so to accommodate growth. |
| 10. | Multi-position thumb which enables thumb to | None |
| | be swung out of the way so fingers can close into | |
| | the palm, or moved up to the side to provide | |
| | lateral pinch against the first finger. | |
| 11. | A complete line of hands ranging from child | This is the standard, accepted |
| | to adult, with progressive features and capability | approach. However, other than |
| | additions based on development and maturation. | hooks, no line offers the same basic |
| | | design features with age-appropriate |
| | | additions that can be utilized from |
| | | childhood to adulthood. All current |
| | | hands on the market incorporate |
| | | significantly different designs and |
| | | cosmetic features over the range from |
| | | child to adult sized hands. |
| 12. | Less costly than the average myoelectric | Body-powered hands are generally |
| | system. Priced comparable to or below popular | much less expensive than complete |
| | body-powered hands and hooks. | myoelectric systems in a comparable |
| | | size. |

The following is a description of the test and verification criteria applicable to the present invention: designed for expandability due to child growth—test metric: normal growth in a child over the time span of four years (from age three to seven) is measurable by lengthening of major bone structures in the arm and hand. The present invention includes expandability to meet this growth expectancy.

Grasp locking capability—test metric: The present invention has complete locking capability sufficient to hold a golf club, baseball bat or fishing rod. As seen from preliminary data, the present invention has the capability to measure the required grasp strength to achieve certain tasks. The present invention includes grasp-locking capability to meet this required holding force.

Individual finger locking capability—test metric: The present invention has individual locking capability sufficient to maintain each finger closed once locked in place. Each finger locking mechanism is able to hold the force created by the return spring force of the finger. The present invention includes individual finger locking capability to overcome the required finger spring force.

Three degree of freedom wrist joint—test metric: The present invention has three degrees of freedom as seen in the human wrist. The present invention includes three degrees of freedom rotation of the wrist comparable to the normal wrist.

Dynamic tensional rotation control of wrist—test metric: It is desired that the patient have control over the frictional tension in the rotation of the wrist joint. The present invention implements a frictional wrist joint with operator control ranging from freely moving wrist joint to tension sufficient to hold objects such as a fishing pole.

Dynamic grasp control to allow grasping of irregular objects—test metric: It is desired that the full hand grasp be able to grasp irregular objects with balanced grasp strength on each finger. The present invention allows the grasping of a baseball with balanced grasp strength across all fingers.

Extended grasp for gripping larger objects such as a soda can—test metric: It is desired that the grasp opening of the prosthesis be enlarged by the user when attempting to grasp objects larger than is possible using the standard prosthetic hand. The present invention allows grasping of an object twice the widest opening width of a standard child prosthesis

The specific embodiments of the present invention include integration of features and improvements in functionality, which are not currently available in any single prosthetic hand/arm. The present invention incorporates the following functional features in a single hand/limb design, including several innovations not currently available in existing devices as described above: designed for expandability to accommodate child growth; grasp locking capability; individual finger locking capability; three degree of freedom wrist joint; dynamic tensional rotation control of wrist; dynamic grasp control to allow grasping of irregular objects; extended grasp for gripping larger objects such as a soda can; excessive force breakaway; and PC based software to support user-adjustment of the prosthesis.

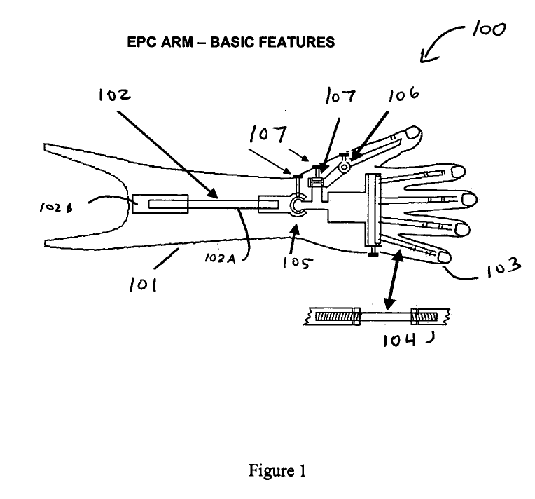

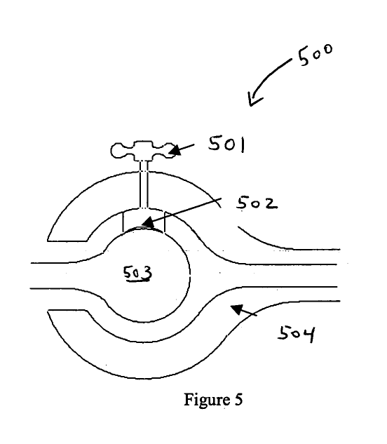

Referring now to the Figures, as seen in FIG. 1, one or more of the features of the present invention may be integrated into a single lower arm and hand prosthesis 100. The designs of the multiple features will first be completed individually and then modified as required while integration is undertaken. The invented device may be fabricated out of an easily machineable materials such as high-density plastic or aluminum. Prosthetic limb 100 includes an extensible forearm 101, the length of which is extensible using a turnbuckle—style threaded sleeve and locknuts, or collet mechanism 102. The forearm can be covered by a flexible glove made of latex or other material to be replaced as the limb is expanded. Fingers 103 of the prosthetic limb 100 are adapted to be lengthened using a turnbuckle style threaded sleeve and locknuts or collet mechanism 104. Fingers 103 can be compliant-grasp fingers with individual control for locking as seen in FIG. 5. Wrist 105 includes dynamic tensional rotation control. Pivot joints 106 provide opposable thumb and grasp extension. Teflon-clutch locking controls 107 provide locking and release functionality.

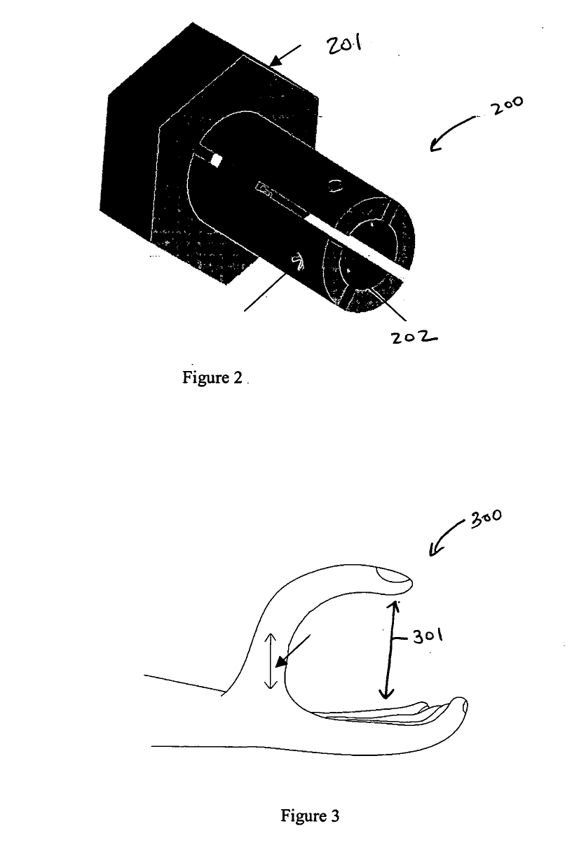

A significant advantage of the present invention is increased useful life of prosthetic arms for children, based upon an easily adjustable extension infrastructure using the retaining tube 200 with retaining nut 201 and locking beads 202 of FIG. 2. Added adjustability will allow a representative of the user such as a medical professional, family member, or care provider, to adjust the length of several structural segments to increase the lifetime of the prosthesis. The present invention increases the time between replacements due to the child's growth. Several embodiments of this can be implemented. Referring back to FIG. 1, all design components of the this aspect of the present invention require three parts: a shaft 102A, a collar and a hollow tube 102B. In all embodiments, the shaft will slide into the tube and be retained by the collar. In one embodiment of the locking capability will be operable through the tightening of the collar, similar to a collet in lathe machining technology. Additional embodiments include locking pieces inside the hollow tube, such that a tightening of the collar results in the increasing force being applied on the solid shaft. All embodiments of this aspect of the present invention will result in a structural member that can be secured in multiple lengths. Furthermore, by loosening the collet mechanism, the shaft can loosely slide into the tube to the new desired length. The collet mechanism can then be retightened, thus locking the member length at its new desired dimension.

Another aspect of the present invention is its ability to grasp an object and hold the grasp without maintaining muscle contraction. This mechanism overcomes one of the primary shortcomings of normally open hands: once the muscle contractive force is removed, the hand opens, thus dropping the object or item that was being held. This mechanism uses a control mechanism for grasp control. Conventional body powered prosthesis use cable control wires, and that aspect remains integral to the present invention. In addition, however, the present invention uses a locking mechanism, which has several embodiments: A locking mechanism using a toggle type mechanical switch which, when in one position, will have no force applied to the control wire, and while in the opposite position, will impart significant force against the control wire. This applied force, in action, locks the cable at its then current position. The position lock ranges from completely open to a completely closed finger, which is defined as thumb touching fingers.

Referring back to FIG. 1, element 104 illustrates an alternative embodiment of the expandability feature of the present invention. This embodiment uses a threaded shaft, either male or female threading, mating to threaded sleeve or sleeves, with the opposite threading. The expandable feature is enabled by screwing the shaft into or out of the sleeve, on one or both ends. This may also be accomplished turnbuckle-style, with opposite threading on each end of the shaft. This embodiment may be utilized for both the forearm component of the present invention, and for the hand components such and fingers and thumbs, and may also be used for the upper-arm segment in the case of an above-elbow amputee. A locknut, or locknuts, then enable the shaft to be fixed in position relative to the sleeve or sleeves.

An aspect of the present invention that is required as a result of other features, such as the locking capability of the hand, is a breakaway. The present invention is thus designed such that the force required to break a hold will be patient or care giver adjustable and provides shock absorption, protecting the upper arm from serious injury. This aspect of the present invention may be accomplished in one embodiment by incorporating a friction-based clutch in the shaft and/or sleeve of the device, such that the clutch will “give” when force above a preset limit is applied to the device.

Further grasping capabilities are enabled using individual finger locking control. Individual finger locking control allows the user to accomplish many different grasp configurations. For instance, if fingers are numbered using the pointing finger as finger 1 and the smallest finger as finger 4, and the thumb, then by closing and locking all fingers and the thumb, one can implement a fist. Similarly, closing the hand and locking fingers two, three and four will result in only finger one and the thumb moving upon removal of muscle control. This will allow two finger grasping capability such as is found in the standard claw type gripper. Similarly, a three-finger chuck gripper can be implemented by locking fingers three and four. Locking fingers two, three and four as well as the thumb results in only the pointer finger returning to straight when muscle control is relaxed, allowing pointing and activation of buttons such as in an elevator.

Additionally it is desired that a prosthetic hand 300 be able to grasp objects larger than the opening of the standard child prosthesis, as seen in FIG. 3. Enhanced functionality can be achieved through a mechanism, which will allow the user the ability to extend the thumb to a second position, such that the hand has a larger opening 301. This will allow the user to grasp an object or item such as a soda can or other larger item. One possible embodiment of this technical innovation is a multi position or dynamic sliding mechanism of the thumb mounting within the hand. In a very basic implementation, this could be accomplished in a similar manner as a pipe wrench. A more technically innovative design of the present invention includes a frictional sliding mechanism on the cross bar which holds the spacing between fingers and thumb.

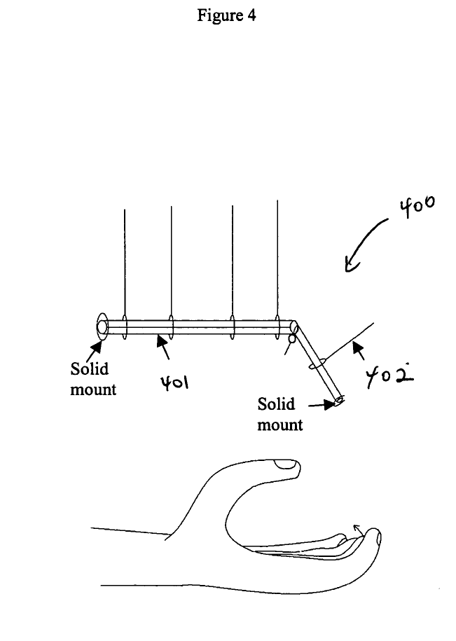

The final grasping aspect of the present invention is a mechanism 400 designed to allow dynamic distributed force grasping. This aspect of the present invention allows all fingers to grasp an object, applying uniform force with each individual finger as seen in FIG. 4. A dynamic grasp allows a user to grasp a baseball with finger contact on all fingers while applying uniform pressure from each finger. In an extreme case, this grasp will allow the user to grasp any irregularly shaped object. As seen in FIG. 4, one embodiment of the dynamic grasp mechanism is implemented using an elastic material 401 for the interconnect structure which holds the finger spacing within the hand. This allows the grasp control wire 402 from the shoulder harness to apply one force, which would be distributed as necessary to each individual finger. For instance, if finger one encountered resistance, then the other fingers would continue to close. The only constant force in this embodiment is the total force applied by the control wire and harness. Another embodiment of this innovation is designed using elastic materials for the control wires to each finger. Thus the elastic would stretch when one finger meets resistance, while allowing the other fingers to continue to close until they meet resistance as well. This aspect of the present invention enables the user an unlimited grasping ability for objects large enough to fit within the hand.

In another aspect of the present invention, the wrist 105 is adapted to rotate fully in three-dimensional space as would a normal human wrist. This will be accomplished through the use of a ball and socket joint 500 as seen in FIG. 5. Modifications on the socket are adapted to allow a frictional control, using tension control 501, Teflon clutch 502 on ball 503 located in socket 504 which permits the user to adjust the “looseness” of the wrist from an external control dial on the housing of the prosthesis. Increased friction permits a stiff wrist as is needed in holding a fishing pole, while reduction in frictional force will allow a lose wrist as is need in swinging a golf club.

The combination of all task-specific mechanisms described herein are incorporated into a state-of-the-art structural housing, which will is covered using conventional prosthesis covering materials. The overall look of the prosthesis will be designed to closely simulate the normal child's arm and hand.

The advantages of the present invention over the prior art include: significantly improved functionality in a lightweight, body-powered, esthetically pleasing, general-purpose prosthesis; expandability in size (up to 30% or more), in order to accommodate a child's growth; affordability by a large percentage of the at-need population, based on achievement of a robust, cost-effective, readily manufacturable design that emphasizes body-power instead of myoelectric control. Because of the present invention's expandability feature, a child should be able to use the same prosthetic hand for a much longer time as compared to conventional devices that require replacement with a new, larger hand; and provision of PC-based tools for interactive patient/parent training, and for software-directed growth adjustments by the patient, parent or care provider.

The embodiments shown and described above are only exemplary. Even though several characteristics and advantages of the present invention have been set forth in the foregoing description together with details of the invention, the disclosure is illustrative only and changes may be made within the principles of the invention to the full extent indicated by the broad general meaning of the terms used in herein and in the attached claims.

1. A prosthetic limb, comprising:
a arm stump attachable first end;
a means at the first end for attachment to an arm stump;
an extensible forearm prosthesis portion; and
a prosthetic hand or gripping device attached to the distal end of the forearm prosthesis portion.

2. The prosthetic limb of claim 1, further comprising being body powered.

3. The prosthetic limb of claim 1, further comprising being body powered.

4. The prosthetic limb of claim 1, further comprising said forearm prosthesis portion being extensible in length by means of an adjustment mechanism integral to the prosthetic arm and adjustable with or without use of tools.

5. The prosthetic limb of claim 1, adapted to be attachable to the arm stump below the elbow.

6. The prosthetic limb of claim 1, adapted to being attachable to the arm stump at a location that otherwise be above the elbow.

7. The prosthetic limb of claim 1, further comprising the prosthetic hand or gripping device being adapted to be locked and unlocked in a range of positions as may be appropriate for grasping or gripping various objects or items.

8. The prosthetic limb of claim 7, further comprising a friction based user controlled tensioning device, adapted to adjust the ease of rotation of said gripping mechanism.

9. The prosthetic limb of claim 1, wherein the prosthetic hand or gripping device is normally open.

10. The prosthetic limb of claim 1, wherein the prosthetic hand or gripping device is normally closed.

11. The prosthetic limb of claim 1, wherein the prosthetic hand or gripping device further comprises a means of release or breakaway in the event of application of force above a preset limit on the held object or item.

12. The prosthetic limb of claim 1, wherein the prosthetic hand or gripping device further comprises the essential form of a human hand, with fingers and/or thumb extensible in length by means of an adjustment mechanism or mechanisms integral to the prosthetic hand or gripping device and adjustable with or without the use of tools.

13. The prosthetic limb of claim 12, wherein the prosthetic hand or gripping device further comprises a means of locking and unlocking individual fingers and thumb in a range of positions.

14. The prosthetic limb of claim 1, further comprising a joint with 3 degrees of freedom attaching the forearm portion of said prosthetic limb to the prosthetic hand or gripping device, said joint forming the wrist of the prosthetic limb.

15. The prosthetic limb of claim 14, further comprising torsion elements in one or more of the 3 degrees of freedom, such that displacement from a normal centered position in any degree of freedom results in a torsion force tending to return the limb to a centered position.

16. The prosthetic limb of claim 15, further comprising a friction based user controlled tensioning device, adapted to adjust ease of rotation of said joint.

17. The prosthetic limb of claim 15, further comprising being adapted to be attachable to the arm stump above the location where there would otherwise be an elbow.

18. The prosthetic limb of claim 15, further comprising being adapted to be attachable to the arm stump below the elbow.

19. The prosthetic limb of claim 1, further comprising adjustment settings relating to the prosthetic limb consisting of one from the group of: markings on the prosthetic arm, settings adjustable by means of software-based algorithms and algorithms based on measurements of body parts of the user.

20. The prosthetic limb of claim 1, further comprising the prosthetic hand or gripping device having a dynamic grasp, such that force exerted upon an object is distributed uniformly between fingers, thumb and palm of the prosthetic hand or gripping device.

21. The prosthetic limb of claim 1, further comprising:
the prosthetic hand or gripping device further comprising a latch feature adapted to allow the prosthetic hand or gripping device to be locked in one of a range of positions while force is maintained on an object or item being gripped; and
the prosthetic hand or gripping device being releasable from its latched position when force is released.

22. The prosthetic limb of claim 1, further comprising a latch feature adapted to allow the wrist joint of the prosthetic arm to be locked in one of a range of positions while force is maintained on an object or item being gripped; and
the wrist joint being releasable from its latched position when force is released.

23. A method of fitting a prosthetic device to a patient, comprising programming a software program with adjustment algorithms based on model-correlated patient specific arm-dexterity parameters to the operational settings and element characteristics.

24. The method of claim 23, further comprising providing patient specific adaptability in terms of customization and growth of patient.
